(19) United States
(12) Patent Application Publication
(10) Pub. No.: US 2019/0289695 A1
(43) Pub. Date: Sep. 19, 2019

(54) BRIGHTNESS ADJUSTMENT DEVICE AND EYE PROTECTION LAMP

(71) Applicants: BOE TECHNOLOGY GROUP CO., LTD., Beijing (CN); HEFEI BOE OPTOELECTRONICS TECHNOLOGY CO., LTD., Anhui (CN)

(72) Inventors: Jiong HUANG, Beijing (CN); Peng LI, Beijing (CN); Wencheng HU, Beijing (CN); Lele HAN, Beijing (CN)

(73) Assignees: BOE TECHNOLOGY GROUP CO., LTD., Beijing (CN); HEFEI BOE OPTOELECTRONICS TECHNOLOGY CO., LTD., Anhui (CN)

(21) Appl. No.: 16/128,848

(22) Filed: Sep. 12, 2018

(30) Foreign Application Priority Data
Mar. 14, 2018 (CN) 201820349278.3

(51) Int. Cl. H05B 37/02

(52) U.S. Cl. CPC H05B 37/0227; A61B 5/024; H05B 37/0272

(57) ABSTRACT

A brightness adjustment device and an eye protection lamp are provided. The brightness adjustment device is applied in the eye protection lamp and includes a physical sign detection assembly, configured to obtain heart rate information of a user, and generate and transmit a brightness adjustment signal based on the heart rate information of the user; and a brightness adjustment assembly, connected to the physical sign detection assembly, and configured to adjust brightness of a light emitting device connected to the brightness adjustment assembly based on the brightness adjustment signal.

### CROSS-REFERENCE TO RELATED APPLICATION

This application claims priority to Chinese Patent Application No. 201820349278.3 filed on Mar. 14, 2018, which is incorporated herein by reference in its entirety.

### TECHNICAL FIELD

The present disclosure relates to the field of an eye protection lamp, in particular to a brightness adjustment device and an eye protection lamp.

### BACKGROUND

Due to insufficient illumination or excessive light at the work place of a user, uneven light distribution or flickering of the light source, eyes of a person may feel glare or reflective, which usually leads to visual fatigue. Visual fatigue is a common syndrome caused by prolonged improper use of the eyes. The symptoms are fatigue, sore eyes, dry eyes, blurred vision, etc., and may be accompanied by symptoms of dizziness and headache. Frequent visual fatigue can cause eyesight to decline and early aging. Visual fatigue is to be avoided to improve visual health.

### SUMMARY

In one aspect, the present disclosure provides in some embodiments a brightness adjustment device, it includes a physical sign detection assembly, configured to obtain heart rate information of a user, and generate and transmit a brightness adjustment signal based on the heart rate information of the user; and a brightness adjustment assembly, connected to the physical sign detection assembly, and configured to adjust brightness of a light emitting device connected to the brightness adjustment assembly based on the brightness adjustment signal.

In some embodiments, the physical sign detection assembly includes a heart rate detection module, configured to detect a heart rate value of the user; and a first controller, connected to the heart rate detection module and configured to generate a brightness adjustment signal based on the received heart rate value.

In some embodiments, the physical sign detection assembly further includes a first communication module, connected to the first controller and configured to transmit the brightness adjustment signal received by the first controller to the brightness adjustment assembly.

In some embodiments, the heart rate detection module comprises a pulse sensor.

In some embodiments, the physical sign detection assembly further comprises a wristband; the heart rate detection module and the first controller are arranged on the wristband.

In some embodiments, the wristband comprises a first wristband and a second wristband that are detachably connected.

In some embodiments, the brightness adjustment assembly includes a second controller, connected to the physical sign detection assembly, configured to receive the brightness adjustment signal and generate a voltage adjustment signal based on the brightness adjustment signal.

In some embodiments, the brightness adjustment assembly includes a second communication module, connected to the physical sign detection assembly and the second controller, and configured to transmit the brightness adjustment signal to the second controller.

In some embodiments, the second communication module 201 comprises at least one of a Zigbee module, a Bluetooth module, a WiFi module or a Near field communication module.

In some embodiments, the heart rate detection module is configured to send the detected heart rate value to the first controller, the first controller determines whether the heart rate value meets a preset condition, and if yes, the brightness adjustment of the light emitting device is not performed; if no, the brightness adjustment of the light emitting device is performed.

In some embodiments, the first controller determines whether the heart rate value is within a preset range, and if yes, the preset condition is met; otherwise, the preset condition is not met; or the first controller determines whether the received heart rate value exceeds a first preset value, and if yes, the preset condition is not met, otherwise the preset condition is met; or the first controller determines whether the received heart rate value is lower than a second preset value, if yes, the preset condition is not met; otherwise, the preset condition is met.

In some embodiments, the preset range is preset range information, or range information generated based on to heart rate values of the user within a preset time period.

In some embodiments, the first controller generates a first signal for reducing brightness when the heart rate value exceeds the first preset value, and generates a second signal for increasing the brightness when the heart rate value is lower than the second preset value.

In some embodiments, the second controller generates a third signal for reducing a voltage of a light emitting device based on the received first signal for reducing the brightness, and generates a fourth signal for increasing the voltage of the light emitting device based on the second signal for increasing the brightness.

In another aspect, an eye protection lamp includes a brightness adjustment device and a light source assembly. The brightness adjustment device includes a physical sign detection assembly, configured to obtain heart rate information of a user, and generate and transmit a brightness adjustment signal based on the heart rate information of the user; and a brightness adjustment assembly, connected to the physical sign detection assembly, and configured to adjust brightness of a light emitting device connected to the brightness adjustment assembly based on the brightness adjustment signal. The light source assembly is connected to the brightness adjustment assembly.

In some embodiments, the light source assembly is disposed in a lamp casing and the brightness adjustment assembly is disposed in the lamp socket.

In some embodiments, the physical sign detection assembly includes a heart rate detection module, configured to detect a heart rate value of the user; a first controller, connected to the heart rate detection module and configured to generate a brightness adjustment signal based on the received heart rate value; and a first communication module, connected to the first controller and configured to transmit the brightness adjustment signal received by the first controller to the brightness adjustment assembly; the brightness adjustment assembly comprises: a second controller, connected to the physical sign detection assembly, configured to receive the brightness adjustment signal and generate a voltage adjustment signal based on the brightness adjustment signal; and a second communication module, connected to the physical sign detection assembly and the second controller, and configured to transmit the brightness adjustment signal to the second controller.

In some embodiments, the second communication module transmits the received brightness adjustment signal to the second controller, the second controller adjusts a resistance value of a varistor based on the received brightness adjustment signal, so as to adjust a voltage value of the light source assembly.

In some embodiments, the light source assembly and the varistor are connected in series.

In some embodiments, the second controller and the varistor are disposed on a second main board.

### BRIEF DESCRIPTION OF THE DRAWINGS

FIG. 1 is a schematic structural diagram of a brightness adjustment device according to an embodiment of the present disclosure;

FIG. 2 is a schematic structural view of a physical sign detection assembly in an embodiment of the present disclosure;

FIG. 3 is a schematic structural diagram of a brightness adjustment method in an embodiment of the present disclosure;

FIG. 4 is a schematic structural view of an eye protection lamp in an embodiment of the present disclosure; and

FIG. 5 is a schematic structural diagram of a brightness adjustment assembly according to an embodiment of the present disclosure.

### DETAILED DESCRIPTION

The specific embodiments of the present disclosure are described in detail below with reference to the accompanying drawings, but are not to be construed as limiting the scope of the present disclosure.

It should be understood that various modifications may be made to the embodiments disclosed herein. Therefore, the above description should not be taken as limiting the present disclosure, but merely as an example of the embodiments. Other modifications within the scope and spirit of the present disclosure will occur to those skilled in the art.

The accompanying drawings, which are included in the specification as a part, together with the summary and the detail description are used to explain the principle of the present disclosure.

These and other features of the present disclosure will become apparent from the following description of the embodiments.

It should also be understood that, although the present disclosure has been described with reference to the specific embodiments thereof, those skilled in the art may definitely implement the present disclosure in some other equivalent ways. These equivalent implements have features defined in the claims and seek for protection by the claims.

The above and other aspects, features and advantages of the present disclosure will become more apparent by combining drawings.

Specific embodiments of the present disclosure are described herein below with reference to the drawings. However, it is understood that the disclosed embodiments are only examples of the present disclosure, which can be implemented in various ways. The well-known and/or repeated functions and structures are not described in detail to avoid unnecessary or redundant details. Therefore, specific structural and functional details are disclosed herein, and are not intended to limit the disclosure, but are merely used as a basis of the claims to teach one skilled in the art to use the disclosure in any suitable specific structures.

The description may use the phrases “in one embodiment”, “in another embodiment”, or “in other embodiments”, which may refer to the same embodiment or one or more of the different embodiments.

The embodiments of the present disclosure are described in detail below with reference to the accompanying drawings. The embodiments of the present disclosure provide a brightness adjustment device, which can detect heart rate information of a user in real time, and correspondingly adjust brightness of a desk lamp or other light source device. Thereby, the eyes of the user can be effectively protected and the visual fatigue can be avoided.

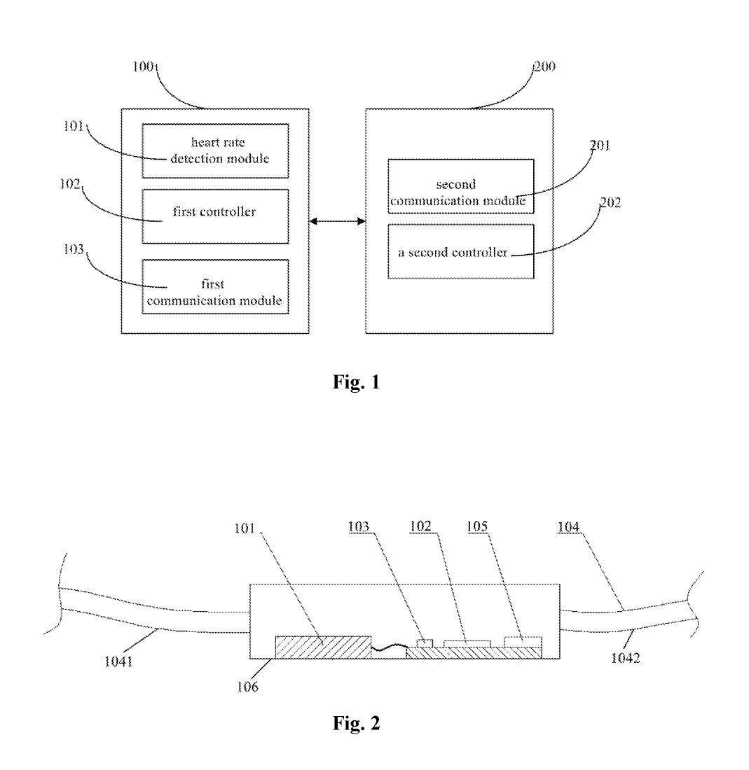

As shown in FIG. 1, it is a schematic structural diagram of a brightness adjustment device according to an embodiment of the present disclosure, wherein the brightness adjustment device can be applied to an eye protection device or other light-emitting device. The light-emitting device may be a display screen, a lamp, etc. The brightness adjustment device may include a physical sign detection assembly 100 and a brightness adjustment assembly 200.

The physical sign detection assembly 100 can receive heart rate information by connecting with other external devices, or can detect the heart rate information of the user to obtain the heart rate information. Moreover, the physical sign detection assembly 100 and the brightness adjustment assembly 200 can be wireless connected or connected with wires for transmitting signals conveniently.

The physical sign detection assembly 100 can generate and transmit a brightness adjustment signal based on the heart rate information of the user. For example, the physical sign detection assembly 100 can include a heart rate detection module 101, a first controller 102, and a first communication module 103.

The heart rate detection module 101 can be configured to detect a heart rate value of the user, and send the detected heart rate value to the first controller 102. The first controller 102 is connected to an output end of the heart rate detecting module 101, and generates a brightness adjustment signal for adjusting the light emitting device based on the received heart rate value. Specifically, in the embodiment of the present disclosure, after receiving the detected heart rate value from the heart rate detection module 101, the first controller 102 may determine whether the heart rate value satisfies a preset condition, and if yes, the brightness adjustment of the light emitting device is not performed; if the preset condition is not met, the corresponding brightness adjustment of the light emitting device is performed. In addition, the first communication module 103 can also receive heart rate information transmitted by other devices, and transmit the received heart rate information to the first controller, thereby generating a brightness adjustment signal by the first controller, and transmitting the brightness adjustment signal to the brightness adjustment assembly 200 through the first communication module 103. The determining, by the first controller, whether the heart rate value meets the preset condition may include: determining, by the first controller, whether the heart rate value is within a preset range, and if yes, the preset condition is met; otherwise, the preset condition is not met; or determining, by the first controller, whether the received heart rate value exceeds the first preset value, and if yes, the preset condition is not met, otherwise the preset condition is met; or determining, by the first controller, whether the received heart rate value is lower than a second preset value, if yes, the preset condition is not met; otherwise, the preset condition is met. The preset range may be preset range information, or may be range information generated according to heart rate values of the user within a preset time.

The preset range information may be a heart rate range inputted by the user, or may be a heart rate range pre-stored in the physical sign detection assembly, and the first preset value and the second preset value may also be inputted by the user. The value may be a preset value pre-stored in the physical sign detecting assembly, and the first controller may conveniently obtain the heart rate range, the first preset value and the second preset value, and perform the above determining operation. In addition, the first controller 102 may acquire a heart rate value within a preset time period, and determine a preset heart rate range, or a first preset value and a second preset value based on the heart rate value by using a preset algorithm. The preset algorithm may be set according to different requirements, and details are not described herein.

Based on the above configuration, the first controller 102 can determine whether the heart rate value of the user meets the preset condition, and further, the brightness adjustment signal can be further generated. For example, the first controller may generate a first signal for reducing the brightness when the heart rate value exceeds the first preset value, and generate a second signal for increasing the brightness when the heart rate value is lower than the second preset value; or generate a first signal for reducing the brightness when the heart rate value exceeds the preset range, and generate a second signal for increasing the brightness when the heart rate value is lower than the preset range.

Further, the above signal may be transmitted to the brightness adjustment assembly 200 through the first communication module 103 communicatively coupled to the first controller 102, so as to implement brightness adjustment. The first communication module 103 may include at least one of a Zigbee module, a Bluetooth module, a WiFi module, an NFC module, and the like.

FIG. 2 is a schematic structural diagram of a physical sign detection assembly 100 according to an embodiment of the present disclosure. The physical sign detection assembly 100 may be constructed as a structure of a wristband, wristwatch, or other wearable assembly. The physical sign detection assembly 100 may further include a wristband 104 and a heart rate detection module 101 connected to the wristband 104, the first controller 102, and the first communication module 103. The heart rate detecting module 101, the first controller 102 and the first communication module 103 can be disposed in a casing, to protect the components therein. The heart rate detection module 101 can acquire a heart rate value by sensing pulse information of the user's wrist. The heart rate detection module in the embodiments of the present disclosure may include a pulse sensor.

The wristband in the embodiment of the present disclosure may include a first wristband 1041 and a second wristband 1042 that are detachably connected, and the first wristband and the second wristband may be oppositely connected to a casing 106. The physical sign detection assembly can be disposed within the casing or on a surface of the casing. Furthermore, the wristband can also be a fixed wristband structure.

In addition, the brightness adjustment assembly 200 in the embodiment of the present disclosure may be connected to the physical sign detection assembly 100, and receive a brightness adjustment signal from the physical sign detection assembly 100, and adjust the voltage of a light emitting device connected to the brightness adjustment assembly 200 based on the received brightness adjustment signal.

Specifically, the brightness adjustment assembly 200 in the embodiment of the present disclosure may include a second communication module 201 and a second controller 202.

The second controller 202 can be connected to the physical sign detection assembly 100, or can be connected to the physical sign detection assembly 100 through the second communication module 201, through which the brightness adjustment signal can be received, and a voltage adjustment signal is generated based on the brightness adjustment signal. The second communication module 201 may include at least one of a zigbee module, a Bluetooth module, a WiFi module, an NFC module, and the like.

Further, the second controller 202 in the embodiment of the present disclosure may generate a third signal for reducing the voltage of the light emitting device according to the received first signal for reducing the brightness of the light, and the second controller 202 may generate a fourth signal for increasing the voltage of the light emitting device based on the second signal for increasing the brightness.

Specifically, the brightness adjustment assembly 200 can adjust the voltage of the light emitting device based on the received brightness adjustment signal. For example, the second controller can adjust the voltage value of the light emitting device based on to the received brightness adjustment signal, thereby adjusting the brightness of the light emitting device. For example, when the first signal is received, the voltage across the light emitting device is reduced, and when the second signal is received, the voltage across the light emitting device is increased, so that the brightness of the light emitting device can be adaptively adjusted when the user's heart rate is abnormal.

Based on the above configuration, the brightness of the light emitting device may be adjusted based on the heart rate of the user in real time, thereby reducing the fatigue state of the user, and also preventing the brightness from further affecting the heart rate parameter of the user.

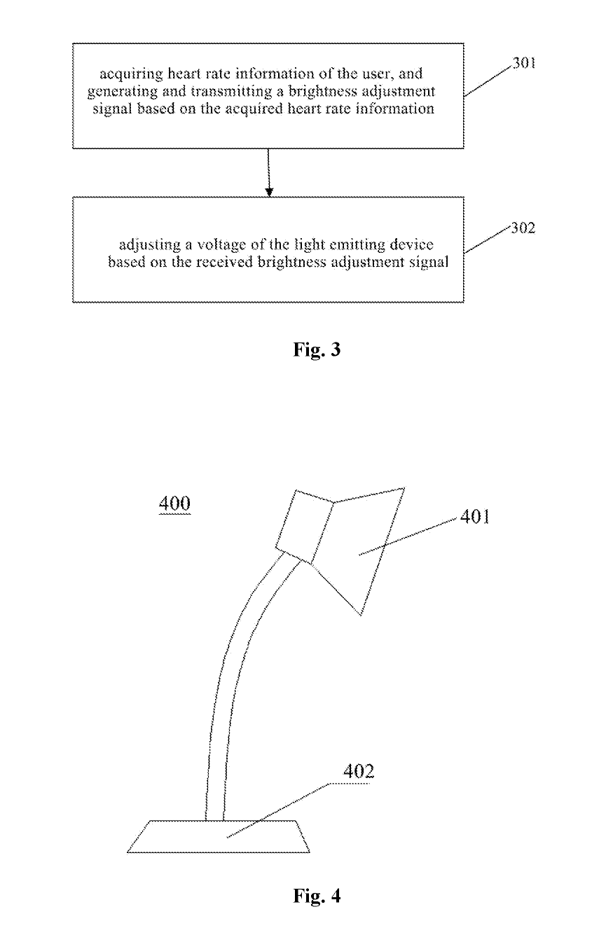

In addition, a brightness adjusting method applied in the brightness adjustment device is provided in the embodiment of the present disclosure, and the principle flow chart of the brightness adjusting method in the embodiment of the present disclosure is shown in FIG. 3. The method includes the following steps.

Step 301, acquiring heart rate information of the user, and generating and transmitting a brightness adjustment signal based on the acquired heart rate information.

Step 302, adjusting a voltage of the light emitting device based on the received brightness adjustment signal.

The physical sign detection assembly 100 in the embodiment of the present disclosure may be configured to detect heart rate information of a user, and generate and transmit a brightness adjusting signal based on the received heart rate information. The physical sign detection assembly 100 can be configured to detect heart rate information of the user, and generate and transmit a brightness adjustment signal based on the received heart rate information. For example, the physical sign detection assembly 100 can include a heart rate detection module 101, a first controller 102, and a first communication module 103.

The heart rate detection module 101 can be configured to detect a heart rate value of the user, and send the detected heart rate value to the first controller 102. The first controller 102 is connected to the output end of the heart rate detection module 101, and generates a brightness adjustment signal for adjusting the brightness of the light emitting device according to the received heart rate value. Specifically, in the embodiment of the present disclosure, after receiving the detected heart rate value from the heart rate detection module 101, the first controller 102 may determine whether the heart rate value meets a preset condition, and if yes, the brightness adjustment of the light emitting device is not performed; if the preset condition is not met, the corresponding brightness adjustment of the light emitting device is performed. The determining, by the first controller, whether the heart rate value meets the preset condition may include: determining, by the first controller, whether the heart rate value is within a preset range, and if yes, the preset condition is met; otherwise, the preset condition is not met; or determining, by the first controller, whether the received heart rate value exceeds the first preset value, and if yes, the preset condition is not met, otherwise the preset condition is met; or determining, by the first controller, whether the received heart rate value is lower than a second preset value, if yes, the preset condition is not met; otherwise, the preset condition is met. The preset range may be preset range information, or may be range information generated based on heart rate values of the user within a preset time period.

Specifically, the generating and transmitting the brightness adjustment signal based on the received heart rate information includes: generating and transmitting a first signal for reducing the brightness when the heart rate value exceeds the first preset value, and generating and transmitting a second signal for increasing the brightness when the heart rate value is lower than the second preset value.

The preset range information may be a heart rate range inputted by the user, or may be a heart rate range pre-stored in the physical sign detection assembly, and the first preset value and the second preset value may also be inputted by the user, or may be preset values pre-stored in the physical sign detection assembly, and the first controller may conveniently obtain the heart rate range, the first preset value and the second preset value, and perform the above determining operation. In addition, the first controller 102 may acquire a heart rate value within a preset time period, and determine a preset heart rate range, a first preset value and a second preset value based on the heart rate value by using a preset algorithm. The preset algorithm may be configured according to different requirements, and details are not described herein.

Based on the above configuration, the first controller 102 can determine whether the heart rate value of the user meets the preset condition, and the brightness adjustment signal can be further generated. For example, the first controller may generate a first signal for reducing the brightness when the heart rate value exceeds the first preset value, and generate a second signal for increasing the brightness when the heart rate value is lower than the second preset value; or generate a first signal for reducing the brightness when the heart rate value exceeds the preset range, and generate a second signal for increasing the brightness when the heart rate value is lower than the preset range.

Further, the above signal may be transmitted to the brightness adjustment assembly 200 through the first communication module 103 communicatively connected to the first controller 102, so as to implement brightness adjustment. The first communication module 103 may include at least one of a Zigbee module, a Bluetooth module, a WiFi module, an NFC module, and the like.

The physical sign detection assembly 100 may be constructed as a structure of a wristband, wristwatch, or other wearable assembly. The physical sign detection assembly 100 may further include a wristband 104 and a heart rate detection module 101 connected to the wristband 104, the first controller 102, and the first communication module 103. The heart rate detecting module 101, the first controller 102 and the first communication module 103 can be disposed in a casing 106, to protect the components therein. The heart rate detection module 101 can acquire a heart rate value by sensing pulse information of the user's wrist. The heart rate detection module in the embodiments of the present disclosure may include a pulse sensor.

The wristband in the embodiment of the present disclosure may include a first wristband 1041 and a second wristband 1042 that are detachably connected, or may also be a fixed wristband structure. In addition, the physical sign detection assembly 100 can also include a battery 105 to provide power to the physical sign detection assembly 100 to ensure proper operation of the physical sign detection assembly.

In addition, the brightness adjustment assembly 200 in the embodiment of the present disclosure may be connected to the physical sign detection assembly 100, and receive a brightness adjustment signal from the physical sign detection assembly 100, and adjust the voltage of light emitting device connected to the brightness adjustment assembly based on the received brightness adjustment signal.

Specifically, the brightness adjustment assembly 200 in the embodiment of the present disclosure may include a second communication module 201 and a second controller 202.

The second controller 202 can be connected to the physical sign detection assembly 100 or can be connected to the physical sign detection assembly 100 through the second communication module 201, through which the brightness adjustment signal can be received, and a voltage adjustment is generated based on the brightness adjustment signal. The second communication module 201 may include at least one of a Zigbee module, a Bluetooth module, a WiFi module, an NFC module, and the like.

Further, the second controller 202 in the embodiment of the present disclosure may generate a third signal for reducing the voltage of the light emitting device according to the received first signal for reducing the brightness of the light, and the second controller 202 may generate a fourth signal for increasing the voltage of the light emitting device based on the second signal for increasing the brightness.

Specifically, the brightness adjustment assembly 200 can adjust the voltage of the light emitting device based on the received brightness adjustment signal. For example, the second controller can adjust the voltage value of the light emitting device based on to the received brightness adjustment signal, thereby adjusting the brightness of the light emitting device. For example, when the first signal is received, the voltage across the light emitting device is reduced, and when the second signal is received, the voltage across the light emitting device is increased, so that the brightness of the light emitting device can be adaptively adjusted when the user's heart rate is abnormal.

Based on the above configuration, the brightness of the light emitting device may be adjusted based on the heart rate of the user in real time, thereby reducing the fatigue state of the user, and also preventing the brightness from further affecting the heart rate parameter of the user.

In addition, an embodiment of the present disclosure may further provide an eye protection lamp, which may include the brightness adjustment device described in the above embodiments. FIG. 4 is a schematic structural view of an eye protection lamp in an embodiment of the present disclosure. The eye protection lamp may include above-described physical sign detection assembly 100, the brightness adjustment assembly 200, and the light source assembly 400. The light source assembly 400 is connected to the brightness adjustment assembly 200.

The brightness adjustment assembly 200 and the light source assembly 400 may be disposed in the lamp body. For example, the light emitting device is disposed on the lamp cover 401, and the brightness adjustment assembly 200 may be disposed in the lamp socket 402. The brightness adjustment assembly 200 and the light source assembly 400 are electrically connected. The physical sign detection assembly 100 is not shown in the drawings and can be connected to the brightness adjustment assembly and transmit the generated brightness adjustment signal.

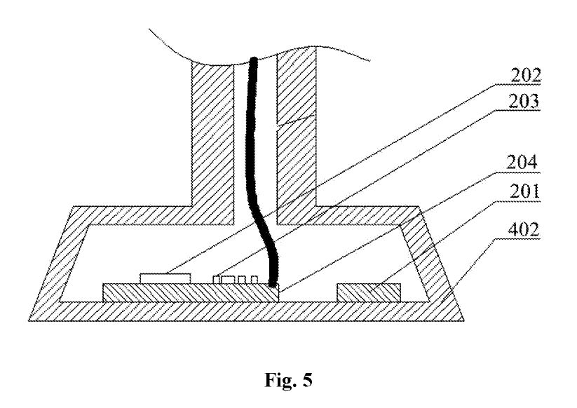

Specifically, FIG. 5 is a schematic diagram of a brightness adjustment assembly in the embodiment of the present disclosure. The second communication module 201 included in the brightness adjustment assembly 200 transmits the received brightness adjustment signal to the second controller 202. The second controller 202 adjusts the resistance value of a varistor 203 according to the received brightness adjustment signal, thereby correspondingly adjusting the voltage value of the light source assembly 400. The light source assembly and the varistor 203 are connected in series. In addition, the second controller 202 and the varistor 203 may be disposed on the second main board 204.

Based on the above configuration, the eye protection lamp of the embodiment of the present disclosure can effectively protect the eyes of the user, prevent visual fatigue, and improve the user experience.

For the convenience and brevity of the description, a person skilled in the art can clearly understand that the electronic device to which the data processing method described above is applied can refer to the corresponding description in the foregoing embodiments, and details are not described herein again.

The above embodiments are for illustrative purposes only, but the present disclosure is not limited thereto. Obviously, a person skilled in the art may make further modifications and improvements without departing from the spirit of the present disclosure, and these modifications and improvements shall also fall within the scope of the present disclosure.

1.-14. (canceled)

15. An eye protection desk lamp, comprising a brightness adjustment device and a light source assembly, wherein
the brightness adjustment device comprises:
a physical sign detection assembly, configured to obtain heart rate information of a user, and generate and transmit a brightness adjustment signal based on the heart rate information of the user; and
a brightness adjustment assembly, connected to the physical sign detection assembly, and configured to adjust brightness of a light emitting device connected to the brightness adjustment assembly based on the brightness adjustment signal,
wherein the light source assembly is connected to the brightness adjustment assembly,
wherein the light source assembly is disposed in a lamp casing and the brightness adjustment assembly is disposed in a lamp socket.

16. (canceled)

17. The eye protection desk lamp according to claim 15, wherein
the physical sign detection assembly comprises:
a heart rate detection module, configured to detect a heart rate value of the user;
a first controller, connected to the heart rate detection module and configured to generate a brightness adjustment signal based on the heart rate value; and
a first communication module, connected to the first controller and configured to transmit the brightness adjustment signal received by the first controller to the brightness adjustment assembly;
the brightness adjustment assembly comprises:
a second controller, connected to the physical sign detection assembly, configured to receive the brightness adjustment signal and generate a voltage adjustment signal based on the brightness adjustment signal; and
a second communication module, connected to the physical sign detection assembly and the second controller, and configured to transmit the brightness adjustment signal to the second controller.

18. The eye protection desk lamp according to claim 17, wherein the second communication module transmits the received brightness adjustment signal to the second controller, the second controller adjusts a resistance value of a varistor based on the received brightness adjustment signal, so as to adjust a voltage value of the light source assembly.

19. The eye protection desk lamp according to claim 18, wherein the light source assembly and the varistor are connected in series.

20. The eye protection desk lamp according to claim 19, wherein the second controller and the varistor are disposed on a second main board.

21. The eye protection desk lamp according to claim 17, wherein the heart rate detection module comprises a pulse sensor.

22. The eye protection desk lamp according to claim 17, wherein the physical sign detection assembly further comprises a wristband, the heart rate detection module and the first controller are arranged on the wristband.

23. The eye protection desk lamp according to claim 22, wherein the wristband comprises a first wristband and a second wristband that are detachably connected.

24. The eye protection desk lamp according to claim 17, wherein the heart rate detection module is configured to send the detected heart rate value to the first controller, the first controller determines the heart rate value meets a preset condition, and the brightness adjustment of the light emitting device is not performed.

25. The eye protection desk lamp according to claim 17, wherein the heart rate detection module is configured to send the detected heart rate value to the first controller, the first controller determines the heart rate value does not meet a preset condition, and the brightness adjustment of the light emitting device is performed.

26. The eye protection desk lamp according to claim 25, wherein the first controller determines whether the heart rate value is within a preset range, and if yes, the preset condition is met; otherwise, the preset condition is not met; or the first controller determines whether the heart rate value exceeds a first preset value, and if yes, the preset condition is not met, otherwise the preset condition is met; or the first controller determines whether the heart rate value is lower than a second preset value, if yes, the preset condition is not met; otherwise, the preset condition is met.

27. The eye protection desk lamp according to claim 26, wherein the preset range is preset range information, or range information generated based on to heart rate values of the user within a preset time period.

28. The eye protection desk lamp according to claim 26, wherein the first controller generates a first signal for reducing brightness when the heart rate value exceeds the first preset value, and generates a second signal for increasing the brightness when the heart rate value is lower than the second preset value.

29. The eye protection desk lamp according to claim 28, wherein the second controller generates a third signal for reducing a voltage of a light emitting device based on the received first signal for reducing the brightness, and generates a fourth signal for increasing the voltage of the light emitting device based on the second signal for increasing the brightness.
